(19) United States
(12) Patent Application Publication
(10) Pub. No.: US 2025/0017565 A1
(43) Pub. Date: Jan. 16, 2025

(54) MEDICAL IMAGING SYSTEM AND CONTROL METHOD THEREOF

(71) Applicant: GE Precision Healthcare LLC, Waukesha, WI (US)

(72) Inventors: Minyu Gu, Wuxi (CN); Zhiwen Wang, Wuxi (CN); Gang Liu, Wuxi (CN); Ke Tao, Wuxi (CN)

(21) Appl. No.: 18/765,763

(22) Filed: Jul. 8, 2024

(30) Foreign Application Priority Data
Jul. 10, 2023 (CN) 202310836420.2

(51) Int. Cl. A61B 8/00; A61B 8/08; G06T 7/00; G06T 7/246

(52) U.S. Cl. CPC A61B 8/4254; A61B 8/0891; A61B 8/4272; A61B 8/4461; G06T 7/0014; G06T 7/248; G06T 2207/10132; G06T 2207/30101

(57) ABSTRACT

A medical imaging system and a control method thereof. The medical imaging system includes: a scanning device, which is used to scan a predetermined site of a subject to undergo detection, to obtain imaging data containing information about the predetermined site; an indication apparatus, which is disposed in the scanning device; and a control apparatus, which performs analysis of a medical image generated on the basis of the imaging data, and controls, on the basis of an analysis result, the indication apparatus to project a light beam to a predetermined surface, so as to form a pattern on the predetermined surface.

### CROSS-REFERENCE TO RELATED APPLICATION

This application claim priority to Chinese Patent Application No. 202310836420.2,which was file on Jul. 10, 2023 at the Chinese Patent Office. The entire contents of the above-listed application are incorporated by reference herein in their entirety.

### TECHNICAL AREA

Examples of the present application relate to the technical field of medical devices, in particular to a medical imaging system and a control method thereof.

### BACKGROUND

A medical imaging system is capable of obtaining an internal tissue image of a subject to undergo detection in a non-intrusive manner. For example, a scanning device of the medical imaging system can scan a predetermined site of a subject to undergo detection, to obtain imaging data containing information about the predetermined site.

A common medical imaging system is, for example, an ultrasound imaging system, a nuclear magnetic resonance imaging (MRI) system, or a computed tomography (CT) scanning system, and so on.

It should be noted that the above introduction of the background is only for the convenience of clearly and completely describing the technical solutions of the present application, and for the convenience of understanding for those skilled in the art.

### SUMMARY

When a scanning device of a medical imaging system is used to scan a predetermined site of a subject to undergo detection, an operator needs to perform a complicated operation on the device, and needs to adjust a relative position relationship between the scanning device and the subject to undergo detection and/or the posture of the scanning device, so as to obtain a good imaging effect. Ultrasound imaging is used as an example for description. An operator needs to look at both a real-time ultrasound image on a screen and the scanning device on a surface of the subject to undergo detection when adjusting the scanning device (for example, a probe). The posture of the scanning device that needs to be adjusted is determined on the basis of the real-time ultrasound image.

The inventors of the present application found that, in a process of operating a scanning device such as a probe, the operator needs to manually determine the position, the posture, a scanning result, and the like of the scanning device, but a corresponding relationship between an ultrasound image representing internal information of the subject to undergo detection and the position of the scanning device is not clear, which makes a scanning process complicated and time-consuming, and affects scanning efficiency and accuracy. Such problems are particularly prominent in a complex scanning scenario.

To resolve at least one technical problem described above or a similar technical problem, examples of the present application provide a medical imaging system and a control method thereof. In the medical imaging system, an indication apparatus on a scanning device can project a light beam onto a predetermined surface to form a pattern corresponding to a result of analyzing a medical image, and the pattern can indicate to an operator how to move the scanning device and/or indicate an examination result. Therefore, a scanning process can be simplified and scanning efficiency and accuracy can be improved.

According to one aspect of the examples of the present application, a medical imaging system is provided. The medical imaging system comprises:
a scanning device, which is used to scan a predetermined site of a subject to undergo detection, so as to obtain imaging data containing information about the predetermined site;
an indication apparatus, which is disposed in the scanning device; and
a control apparatus, which performs analysis of a medical image generated on the basis of the imaging data, and controls, according to an analysis result, the indication apparatus to project a light beam to a predetermined surface, so as to form a pattern on the predetermined surface.

According to another aspect of the examples of the present application, a control method of a medical imaging system is provided. The medical imaging system comprises:
a scanning device, which is used to scan a predetermined site of a subject to undergo detection, so as to obtain imaging data containing information about the predetermined site;
an indication apparatus, which is disposed in the scanning device; and a control apparatus, and
the control method comprises:
the control apparatus performs analysis of a medical image generated on the basis of the imaging data, and controls, according to an analysis result, the indication apparatus to project a light beam to a predetermined surface, so as to form a pattern on the predetermined surface.

One of the beneficial effects of the examples of the present application is that: The indication apparatus on the scanning device can project a light beam onto a predetermined surface to form a pattern corresponding to a result of analyzing a medical image, and the pattern can indicate to an operator how to move the scanning device and/or indicate an examination result. Therefore, the operator does not need to perform complicated judgment on operations such as translation and rotation of the scanning device and/or the examination result, and a scanning process is simplified, thereby improving scanning efficiency. In addition, the operator can increase the number of sites to be scanned for a unit of time, thereby improving scanning accuracy.

With reference to the following description and drawings, specific embodiments of the examples of the present application are disclosed in detail, and the means by which the principles of the examples of the present application can be employed are illustrated. It should be understood that the embodiments of the present application are therefore not limited in scope. Within the scope of the spirit and clauses of the appended claims, the embodiments of the present application include many changes, modifications, and equivalents.

### BRIEF DESCRIPTION OF THE DRAWINGS

The included drawings are used to provide further understanding of the examples of the present application, which constitute a part of the description and are used to illustrate the embodiments of the present application and explain the principles of the present application together with textual description. Evidently, the drawings in the following description are merely some examples of the present application, and a person of ordinary skill in the art may obtain other embodiments according to the drawings without involving inventive skill. In the drawings:

FIG. 1 is a schematic diagram of a medical imaging system according to an example of the present application;

FIG. 2 is a schematic diagram of a scanning device 1 according to the present application;

FIG. 3 is a schematic diagram of a use scenario according to the present application;

FIG. 4 is a schematic diagram of a pattern indicating first target translation information;

FIG. 5 is a schematic diagram of a pattern indicating second target rotation information;

FIG. 6 is a schematic diagram of the posture of a scanning device 1 relative to a predetermined site 11;

FIG. 7 is a schematic diagram of adjusting a scanning position of a scanning device 1;

FIG. 8 is a schematic diagram of an ultrasound imaging system according to an example of the present application; and

FIG. 9 is a schematic diagram of a control method of a medical imaging system according to an example of the present application.

### DETAILED DESCRIPTION

The foregoing and other features of the examples of the present application will become apparent from the following description and with reference to the drawings. In the description and drawings, specific embodiments of the present application are disclosed in detail, and part of the embodiments in which the principles of the examples of the present application may be employed are indicated. It should be understood that the present application is not limited to the described embodiments. On the contrary, the examples of the present application include all modifications, variations, and equivalents which fall within the scope of the appended claims.

In the examples of the present application, the terms “first” and “second” and so on are used to distinguish different elements from one another by their title, but do not represent the spatial arrangement, temporal order, or the like of the elements, and the elements should not be limited by said terms. The term “and/or” includes any one of and all combinations of one or more associated listed terms. The terms “comprise”, “include”, “have”, etc., refer to the presence of stated features, elements, components, or assemblies, but do not exclude the presence or addition of one or more other features, elements, components, or assemblies. The terms “pixel” and “voxel” may be used interchangeably.

In the examples of the present application, the singular forms “a” and “the” or the like include plural forms, and should be broadly construed as “a type of” or “a kind of” rather than being limited to the meaning of “one”. Furthermore, the term “the” should be construed as including both the singular and plural forms, unless otherwise specified in the context. In addition, the term “according to” should be construed as “at least in part according to . . . ”, and the term “based on” should be construed as “at least in part based on . . . ”, unless otherwise clearly specified in the context.

The features described and/or illustrated for one embodiment may be used in one or more other embodiments in an identical or similar manner, combined with features in other embodiments, or replace features in other embodiments. The term “include/comprise” when used herein refers to the presence of features, integrated components, steps, or assemblies, but does not exclude the presence or addition of one or more other features, integrated components, steps, or assemblies.

### Examples

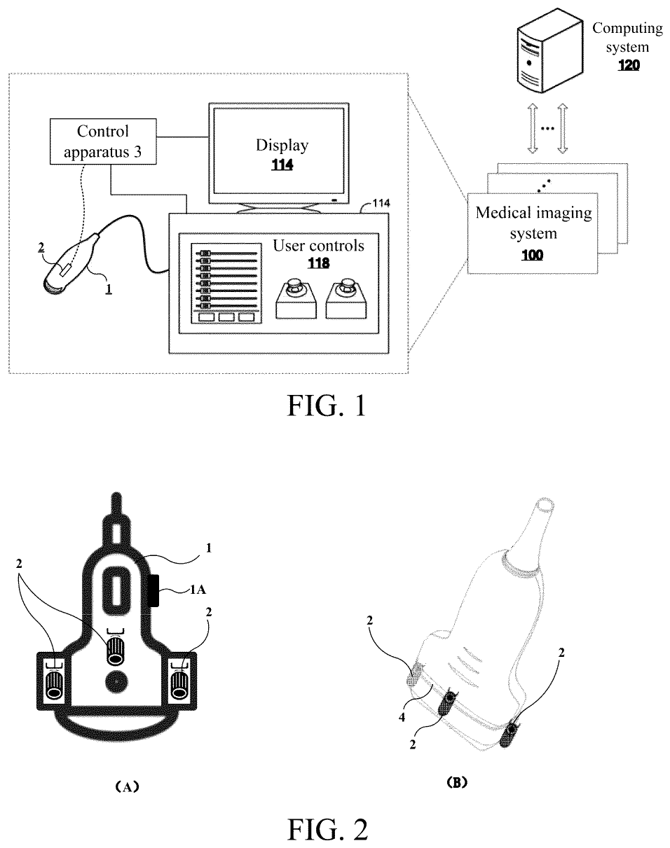

An example of the present application provides a medical imaging system. FIG. 1 is a schematic diagram of the medical imaging system according to this example of the present application. As shown in FIG. 1, the medical imaging system 100 includes: a scanning device 1, an indication apparatus 2, and a control apparatus 3.

The scanning device 1 may be used to scan a predetermined site of a subject to undergo detection, so as to obtain imaging data containing information about the predetermined site; the indication apparatus 2 may be disposed in the scanning device 1; and the control apparatus 3 performs analysis of a medical image generated on the basis of the imaging data, and controls, according to an analysis result, the indication apparatus 2 to project a light beam to a predetermined surface, to form a pattern 21 on the predetermined surface 2.

In the present application, the pattern 21 formed on the basis of the analysis result of the medical image can indicate to an operator how to move the scanning device and/or indicate an examination result. Therefore, the operator does not need to perform complicated judgment on operations such as translation and rotation of the scanning device and/or the examination result by observing an ultrasound image in combination with experience of the operator, but only needs to focus on a pattern near a probe to know how to operate the probe. On the one hand, the line of sight is not diverted to a display, and on the other hand, an indication of movement is more intuitive. Thus, a scanning process is simplified, thereby improving scanning efficiency. In addition, the operator can increase the number of sites to be scanned for a unit of time, thereby improving scanning accuracy.

In the present application, the medical imaging system 100 is, for example, an ultrasound imaging system, a nuclear magnetic resonance imaging (MRI) system, or a computed tomography (CT) scanning system. In the following descriptions of the present application, that the medical imaging system 100 is an ultrasound imaging system is used as an example for description, but contents of these descriptions are not limited to the ultrasound imaging system and can also be applied to another type of medical imaging system.

In the medical imaging system 100 as the ultrasound imaging system, the scanning device 1 includes, for example, a probe, and the scanning device I can emit an ultrasound wave to a predetermined site of a subject to undergo detection and receive an echo of the ultrasound wave, thereby obtaining imaging data. The imaging data may be used to generate a medical image. A current medical image obtained by scanning refers to a medical image (an anatomical image of a specific section) that can reflect a state (form) of the predetermined site (for example, an organ or a tissue such as a blood vessel or a heart) of the subject to undergo detection at a current time (in real time).

FIG. 2 is a schematic diagram of a scanning device I according to the present application. As shown in FIG. 2, an indication apparatus 2 may be disposed in the scanning device 1. In at least one example, as shown in (A) in FIG. 2, the indication apparatus 2 may be integrated into the scanning device 1. In at least another example, as shown in (B) in FIG. 2, the indication apparatus 2 may be installed on the scanning device 1 by means of a connecting means 4. For example, the connecting means 4 may be sleeved on a housing of the scanning device 1, and the indication apparatus 2 is installed on the connecting means 4, thereby arranging the indication apparatus 2 on the scanning device 1. In addition, the connecting means 4 may be detached from the scanning device 1, to disengage the indication apparatus 2 from the connecting means 4.

In some examples, the indication apparatus 2 may be a matrix of light emitting elements, for example, a matrix of light emitting diodes. Therefore, the indication apparatus 2 can form a required pattern on a predetermined surface.

In FIG. 2, there are three indication apparatuses 2, wherein one indication apparatus 2 is disposed on one side (for example, the left side) of the scanning device 1 in the length direction, one indication apparatus 2 is disposed on the other side (for example, the right side) of the scanning device 1 in the length direction, and one indication apparatus 2 is disposed on one side (for example, the front side) of the scanning device 1 in the width direction. The present application may not be limited thereto, for example, the three indication apparatuses 2 may also be disposed at other positions of the scanning device 1. The number of the indication apparatuses 2 is not limited to three, for example, the number may be one or two or more than three.

In the present application, the indication apparatus 2 projects a light beam onto the predetermined surface, wherein the predetermined surface may be a surface of a subject to undergo detection. For example, the subject to undergo detection is a human body or another animal body, and the surface of the subject to undergo detection may be a skin surface of the human body or the another animal body, or the like. Since the scanning device 1 usually needs to move on the surface of the subject to undergo detection, such an arrangement manner can make an indication effect of the light beam projected by the indication apparatus 2 more intuitive. That is, a projected pattern can thus directly represent a target movement direction and/or position. Therefore, intuitiveness and efficiency of indication are further improved.

As shown in FIG. 2, the scanning device 1 has a button 1A. An operator of the scanning device 1 may press the button 1A or release the button 1A, thereby generating a signal corresponding to the pressing or releasing. A control apparatus 3 can detect the signal corresponding to the pressing or releasing, and perform corresponding processing.

The control apparatus 3 can communicate with the scanning device 1 and the indication apparatus 2 in a wired or wireless manner, so that the control apparatus 3 can perform analysis of a medical image generated on the basis of imaging data and control, on the basis of an analysis result, the indication apparatus 2 to generate a pattern. In addition, the control apparatus 3 can detect the signal corresponding to pressing or releasing the button 1A, and perform corresponding processing.

As described above in the present application, an embodiment of the present application will have a more obvious effect of improving scanning efficiency in a complex scanning scenario. In the following, the present application is described with reference to a specific use scenario of the medical imaging system 100.

The inventors found that when performing a lower extremity deep vein thrombosis (LDVT) examination using an ultrasound imaging system, an operator (for example, a physician) needs to perform transverse scanning and pressing test on a lower extremity deep vein by using a probe every 2-3 cm, and sometimes also needs to rotate the probe to obtain a high-quality cross-sectional image of the vein. In addition, when the operator adjusts the probe with one hand, the operator sometimes also needs to perform another operation such as parameter adjustment with the other hand.

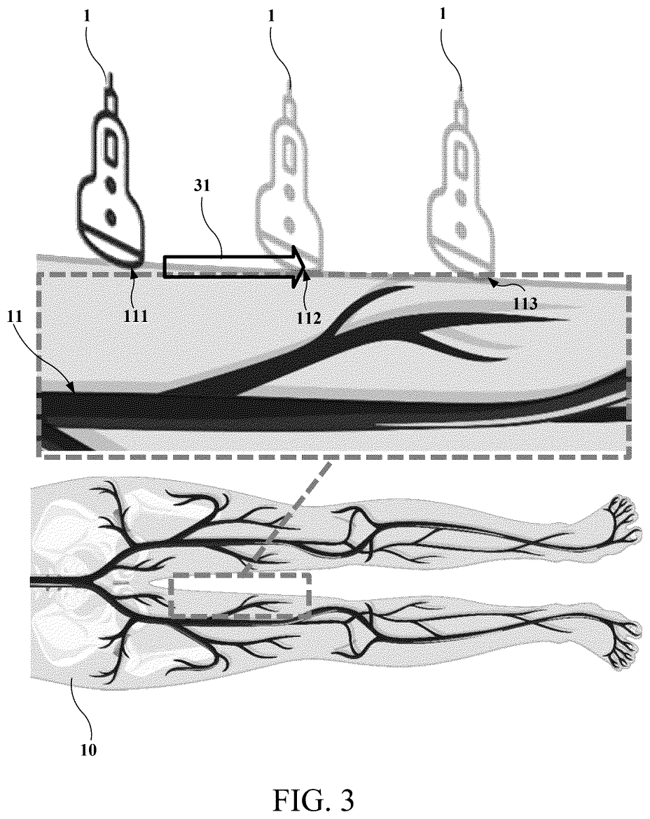

FIG. 3 is a schematic diagram of this scenario. As shown in FIG. 3, in this scenario, a predetermined site 11 of a subject 10 to undergo detection is scanned by the scanning device 1, wherein the subject 10 to undergo detection may be a human body or another animal body, and the predetermined site 11 may be a blood vessel of the subject to undergo detection, for example, an LDVT.

As shown in FIG. 3, the operator may move the scanning device 1 to different positions of the subject 10 to undergo detection, for example, a position 111, a position 112, and a position 113, and perform scanning at each position to obtain imaging data containing information about the predetermined site. The imaging data can be used to generate a medical image.

In the present application, the control apparatus 3 may perform analysis of the medical image, and the analysis includes analysis of a difference between an image of the predetermined site in the medical image and a target. The control apparatus 3 may cause, according to the analysis result, a pattern of a light beam projected by the indication apparatus 2 to indicate target movement information of the scanning device 1, and when the operator moves the scanning device 1 according to the target movement information, the difference can be reduced.

In some examples, the difference includes an offset of the image of the predetermined site 11 relative to a central position of the medical image, and the target movement information includes first target translation information for reducing the offset, wherein the first target translation information includes at least a first target translation direction of the scanning device 1. In addition, the first target translation information may further include a moving distance of the scanning device 1 in the first target translation direction.

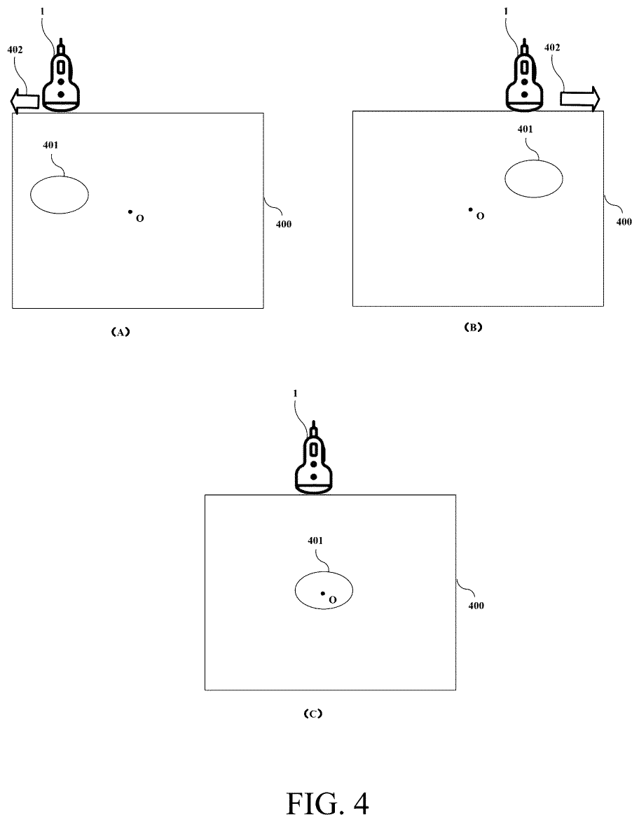

FIG. 4 is a schematic diagram of a pattern indicating first target translation information.

As shown in (A) in FIG. 4, when a scanning device 1 is at the current position, an image 401 of a predetermined site 11 is offset to the left side relative to a central position O (for example, a central position in a transverse direction) of a medical image 400. Therefore, a control apparatus 3 controls an indication apparatus 2 to project a light beam onto the skin of a subject 10 to undergo detection, so as to form a pattern 402 indicating movement of the scanning device 1 to the left side (i.e., a first target translation direction of the scanning device 1). The pattern may be an arrow toward the left.

In addition, the control apparatus 3 may also calculate, according to a mapping relationship between a translation distance of the scanning device 1 and a moving distance of the image 401 of the predetermined site 11 in the medical image 400, a distance that the scanning device 1 moves to the left (i.e., a moving distance in the first target translation direction), and set the length of the pattern 402 according to the distance (for example, the length of the pattern 402 is equal to the distance). Therefore, the pattern 402 can indicate the moving distance in the first target translation direction.

In the case shown in (A) in FIG. 4, an operator moves the scanning device 1 according to the indication of the pattern 402, so that the image 401 of the predetermined site 11 is located at the center in the transverse direction of the medical image 400 (as shown in (C) in FIG. 4). Therefore, a high-quality image 401 can be obtained.

As shown in (B) in FIG. 4, when the scanning device 1 is at the current position, the image 401 of the predetermined site 11 is offset to the right side relative to the central position O (for example, the central position in the transverse direction) of the medical image 400. Therefore, the control apparatus 3 controls the indication apparatus 2 to project a light beam onto the skin of the subject 10 to undergo detection, so as to form the pattern 402 indicating movement of the scanning device 1 to the right side (i.e., the first target translation direction of the scanning device 1). The pattern may be an arrow toward the right.

In addition, the control apparatus 3 may also calculate, according to the mapping relationship between the translation distance of the scanning device 1 and the moving distance of the image 401 of the predetermined site 11 in the medical image 400, a distance that the scanning device 1 moves to the right (i.e., a moving distance in the first target translation direction), and set the length of the pattern 402 according to the distance (for example, the length of the pattern 403 is equal to the distance). Therefore, the pattern 402 can indicate the moving distance in the first target translation direction.

In the case shown in (B) in FIG. 4, an operator moves the scanning device 1 according to the indication of the pattern 402, so that the image 401 of the predetermined site 11 is located at the center in the transverse direction of the medical image 400 (as shown in (C) in FIG. 4). Therefore, a high-quality image 401 can be obtained.

In some examples, the difference includes a difference between a size difference, between a first size in a first direction and a second size in a second direction of the image of the predetermined site 11, and a second threshold, and the target movement information includes second target rotation information for reducing the difference, wherein the second target rotation information includes at least a second target rotation direction of the scanning device 1, for example, clockwise rotation or counterclockwise rotation. In addition, the second target rotation information may further include a rotation angle or a rotation position of the scanning device 1 in the second target rotation direction.

The first direction is, for example, a transverse or horizontal direction of the medical image, and the second direction is, for example, a longitudinal or vertical direction of the medical image. In addition, the present application is not limited thereto, and the first direction may also be, for example, a direction forming a first included angle (for example, the first included angle is not equal to 0 degrees, 90 degrees, or the like) with the transverse or horizontal direction of the medical image, and the second direction may also be, for example, a direction forming a second included angle (for example, the second included angle is not equal to 0 degrees, 90 degrees, or the like) with the longitudinal or vertical direction of the medical image.

The size difference between the first size and the second size may be represented, for example, as a ratio of the first size to the second size, or a difference value between the first size and the second size.

The difference between the size difference and the second threshold may be, for example: an absolute value of the difference between the size difference and the second threshold, or a ratio of the size difference to the second threshold.

The parameters such as the difference value/the threshold may be predefined in advance by an operator according to different subjects to undergo detection. Alternatively, the parameters may be automatically determined by the medical imaging system.

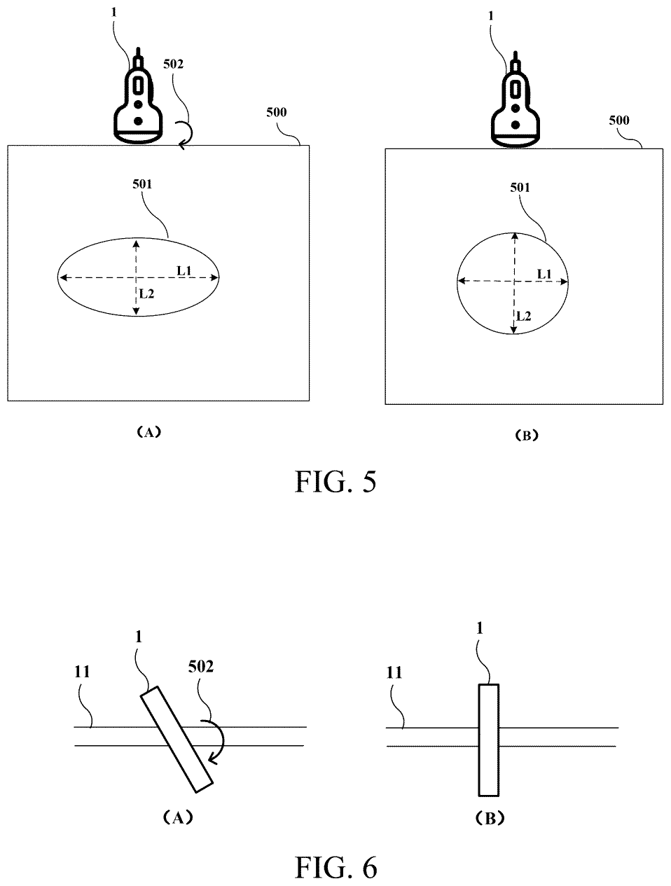

FIG. 5 is a schematic diagram of a pattern indicating second target rotation information, and FIG. 6 is a schematic diagram of the posture of a scanning device 1 relative to a predetermined site 11.

As shown in (A) in FIG. 5, when a scanning device 1 is at the current position, a ratio of a first size LI of an image 501 of a predetermined site 11 in a first direction (for example, a horizontal direction) to a second size L2 in a second direction (for example, a vertical direction) is L1/L2, and L1/L2 is greater than a second threshold (for example, the second threshold is 1). Therefore, a control apparatus 3 controls an indication apparatus 2 to project a light beam onto the skin of a subject 10 to undergo detection, so as to form a pattern 502 indicating rotation of the scanning device 1 in a clockwise direction (i.e., a second target rotation direction of the scanning device 1). The pattern may be an arrow pointing in the clockwise direction.

In addition, the control apparatus 3 may calculate, according to a mapping relationship between a rotation angle of the scanning device 1 and L1/L2 in the image 501 of the predetermined site 11, a rotation angle or rotation position of the scanning device 1 in the clockwise direction (i.e., a rotation angle or rotation position in the second target rotation direction), and set the length of the pattern 502 (for example, the angle of rotation of a central angle corresponding to the length of the pattern 502) according to the rotation angle or rotation position.

(A) in FIG. 5 corresponds to a case in which the length direction of the scanning device 1 is not perpendicular to an extending direction of the predetermined site 11 (for example, an extending direction of a blood vessel), as shown in (A) in FIG. 6. As shown in (A) in FIG. 6, the pattern 502 is an arrow pointing in the clockwise direction, so that an operator can be guided to rotate the scanning device 1, to make the length direction of the scanning device 1 to be perpendicular to the extending direction of the predetermined site 11 (for example, the extending direction of the blood vessel).

In a case shown in (B) in FIG. 5, the operator moves (for example, rotates) the scanning device 1 according to an indication of the pattern 502, so that L1/L2 of the image 501 of the predetermined site 11 reaches the second threshold (as shown in (B) in FIG. 5). Therefore, a high-quality image 501 can be obtained.

(B) in FIG. 5 corresponds to a case, shown in (B) in FIG. 6, in which the operator rotates the scanning device 1 according to the indication of the pattern 502, so that the length direction of the scanning device 1 is perpendicular to the extending direction of the predetermined site 11 (for example, the extending direction of the blood vessel).

In the present application, the control apparatus 3 can perform real-time analysis in a process of moving (for example, translating and/or rotating) the scanning device 1, and control, according to a real-time analysis result, the indication apparatus 2 to form a real-time pattern.

For example, while the operator moves the scanning device 1 (for example, translating the scanning device 1), when the offset of the image 401 relative to the central position O of the medical image 400 increases, the length of the pattern 402 increases according, and when the offset of the image 401 less relative to the central position O of the medical image 400 decreases, the length of the pattern 402 decreases according until the pattern 402 disappears. For another example, while the operator moves the scanning device 1 (for example, rotating and translating the scanning device), when a difference between L1/L2 of the image 501 and the second threshold increases, the length of the pattern 502 increases accordingly, and when the difference between L1/L2 of the image 501 and the second threshold decreases, the length of the pattern 502 decreases accordingly until the pattern 502 disappears.

In the present application, the analysis performed by the control apparatus 3 on the medical image may also include: performing detection on the medical image. The control apparatus 3 may cause, according to a detection result obtained by performing detection on the medical image, a pattern of a light beam projected by the indication apparatus 2 to indicate the detection result, so that the operator can easily confirm and identify the detection result.

When the predetermined site 11 is a blood vessel, the detection performed on the medical image may be an analysis of the health condition of the blood vessel, and the pattern may indicate the health condition as the detection result.

For example, when the detection result is a first result, the pattern is a first pattern, when the detection result is a second result, the pattern is a second pattern, and the first pattern and the second pattern are different in at least one of shape, color, and position. In one specific example, the pattern is a green dot when the analysis of the health condition of the blood vessel indicates that the blood vessel is healthy (for example, there is no thrombus in the blood vessel), the pattern is a red dot when the analysis of the health condition of the blood vessel indicates that the blood vessel is unhealthy (for example, there is a thrombus in the blood vessel), and the like.

In the present application, the detection performed by the control apparatus 3 on a medical image may be one of or a combination of two or more of detection on the basis of a color flow mode image, detection on the basis of a Doppler mode image, detection on the basis of a B mode image, or detection on the basis of elastography.

The detection on the basis of the color flow mode image includes, for example: using a segmentation model (for example, a deep learning model) to obtain a bounding box of a blood vessel (for example, a vein), wherein the bounding box is an ROI of the color flow mode image; and in a color flow mode, using the segmentation model to obtain the area of the vein and the area of a color flow portion, wherein if the area of the color flow portion is very small or zero relative to the area of the vein, it indicates that there may be a thrombus at the detection position.

The detection on the basis of the Doppler mode image includes, for example: In a Doppler mode, if a blood flow velocity drops greatly, it indicates that there may be a thrombus at the detection position.

The detection on the basis of elastography includes, for example: using the segmentation model (for example, the deep learning model) to obtain a vein region; and obtaining, on the basis of an elastography technology, an elasticity value of the vein region and an elasticity value of another part in a medical image obtained after the end of an operation of pressing, by the scanning device 1, the subject to undergo detection, wherein if the elasticity value of the vein region and the elasticity value of the another part, it means that the vein cannot contract, and there may be a thrombus at the detection position.

In the above-described manner, the pattern can provide an intuitive indication to an operator prompt, thereby minimizing diversion of the operator's attention from the scanning device 1 and further improving scanning efficiency. An indication effect of the pattern may be actually understood as a significant change corresponding to a detection result in an ultrasound image. A specific health condition analysis may be determined by the operator by further observing the image as required.

The detection on the basis of the B mode image includes, for example: For a first moment at which the operator starts pressing the subject to undergo detection with the scanning device 1 and for a second moment at which the operator stops pressing the subject to undergo detection with the scanning device 1, the control apparatus 3 may perform detection on the basis of the medical image (for example, a B mode medical image) at the first moment and the medical image (for example, a B mode medical image) at the second moment. In at least one specific example, the control apparatus 3 may obtain a first bounding box of the vein in the medical image at the first moment using the segmentation model (for example, the deep learning model), and obtain a second bounding box of the vein in the medical image at the second moment using the segmentation model (for example, the deep learning model), and if a difference between the height of the first bounding box and the height of the second bounding box is less than a threshold, it indicates that the vein cannot be normally flattened, and there may be a thrombosis at the detection position. In addition, the detection performed by the control apparatus 3 on the basis of the medical image at the first moment and the medical image at the second moment may alternatively be another type of detection.

In the present application, the control apparatus 3 may determine the first moment and second moment on the basis of pressing and releasing, by the operator, the button 1A of the scanning device 1 in (A) in FIG. 2.

For example, the operator may press the button 1A at a start moment of pressing the subject to undergo detection with the scanning device 1; and the operator may release the button 1A at an end moment of pressing the subject to undergo detection with the scanning device 1.

In some examples, the control apparatus 3 may be configured to: determine a moment at which the button 1A is pressed as a first moment at which the scanning device 1 starts pressing the subject 10 to undergo detection; and determine a moment at which the button 1A is released as a second moment at which the scanning device 1 stops pressing the subject 10 to undergo detection.

In some examples of the present application, the detection performed by the control apparatus 3 on a medical image may be one of or a combination of two or more of detection on the basis of a color flow mode image, detection on the basis of a Doppler mode image, detection on the basis of a B mode image, or detection on the basis of elastography. For example, when a thrombus in a vein is detected on the basis of any of the detection methods described above, a corresponding medical image is stored and it is determined that the thrombus is present in the vein.

The foregoing describes a detection method by using an example in which detection is performed to determine whether a thrombus is present in a vein. This application is not limited thereto. The foregoing detection may also be detection for another purpose, and another detection method may be used.

In the present application, after the detection performed on the medical image is completed, the control apparatus 3 further controls the indication apparatus 2 to project a light beam to the predetermined surface, so as to form a movement indication pattern on the predetermined surface. Thus, the operator can move the scanning device 1 to a next position on the subject 10 to undergo detection according to the movement indication pattern.

The movement indication pattern may indicate second target translation information of the scanning device 1, wherein the second target translation information may include a second target translation direction. In addition, the second target translation information may further include a second target translation distance, for example, an end point of the movement indication pattern in the second target translation direction may be used to indicate a target position for the scanning device 1 moving in the second target translation direction.

For example, the scanning device 1 scans the predetermined site 11 at a position 111 to obtain imaging data, the control apparatus 3 performs detection on a medical image generated on the basis of the imaging data, and after the detection is completed (for example, a detection result is a first result or a second result), the indication apparatus 2 projects a light beam onto a predetermined surface (for example, the skin of the subject 10 to undergo detection), to form a movement indication pattern 31 shown in FIG. 3, wherein the movement indication pattern 31 may point to a next position (for example, to the right), and a right end point 311 of the movement indication pattern 31 may be a target position of a target position for the scanning device 1 moving in the second target translation direction, for example, a position of the right end point 311 of the movement indication pattern 31 is a position 112.

In some examples of the present application, when the control apparatus 3 controls the indication apparatus 2 to project a light beam onto the predetermined surface, so as to form a pattern on the predetermined surface, the control apparatus 3 may further control a sound emitting apparatus (not shown) to emit a prompt tone corresponding to the pattern, wherein the prompt tone may be a speech or sound of predetermined content. Therefore, both the pattern and the prompt tone together can provide an indication to the operator, thereby improving reliability and convenience.

For example, when the pattern 402 shown in FIG. 4 is generated, the sound emitting apparatus emits a prompt tone corresponding to the first target translation information; and/or, when the pattern 502 shown in FIG. 5 is generated, the sound emitting apparatus emits a prompt tone corresponding to the second target rotation information; and/or, when the first pattern or the second pattern is generated on the basis of the detection result, the sound emitting apparatus emits a prompt tone corresponding to the first pattern or the second pattern; and/or, when the movement indication pattern 31 shown in FIG. 3 is generated, the sound emitting apparatus emits a prompt tone corresponding to the movement indication pattern 31.

In some examples of the present application, the control apparatus 3 may adjust, according to an offset amount of an image of the predetermined site 10 relative to the center of the image in a second direction, a position that is in the subject 10 to undergo detection and that corresponds to the imaging data obtained by the scanning device 1, to reduce the offset amount. Therefore, a scanning position of the scanning device 1 can be automatically adjusted, and detection accuracy can be improved. The second direction is, for example, a longitudinal or vertical direction of the medical image.

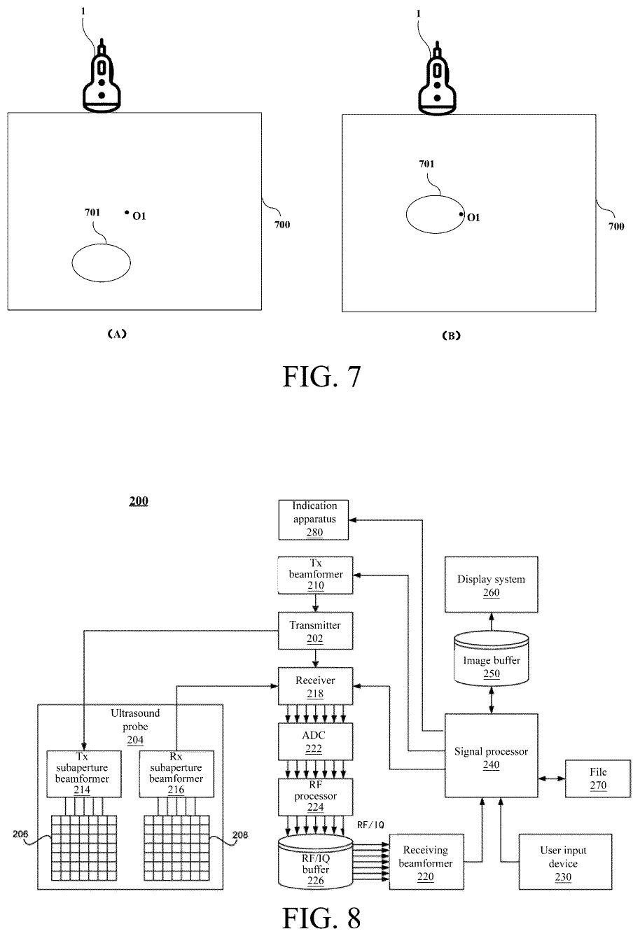

FIG. 7 is a schematic diagram of adjusting a scanning position of a scanning device 1.

As shown in (A) in FIG. 7, when the scanning device 1 is at a current position, the scanning device scans a predetermined site 11 to obtain imaging data, and a medical image 700 is obtained on the basis of the imaging data. The control apparatus 3 may process the medical image 700 to identify an image 701 of the predetermined site 11, for example, the control apparatus 3 uses a deep learning segmentation model to process the medical image 700.

As shown in (A) in FIG. 7, the image 701 of the predetermined site 11 is offset downward relative to a central position O1 (for example, a central position in a longitudinal direction) of the medical image 700. In this way, the control apparatus 3 performs control to adjust a position that is in the subject 10 to undergo detection and that corresponds to the imaging data obtained by the scanning device 1, so as to adjust a position of the image 701 of the predetermined site 11 in a second direction (for example, a longitudinal direction) of the medical image 700. For example, when the scanning device 1 is an ultrasound probe, the control apparatus 3 adjusts a depth of transmission of an ultrasound wave and/or a depth of reception of an ultrasound wave of the scanning device 1. Specifically, the control apparatus 3 can adjust the depth of transmission of an ultrasound wave and/or the depth of reception of an ultrasound wave of the scanning device 1 to move downward.

As shown in (B) in FIG. 7, after the adjustment, the image 701 of the predetermined part 11 is moved upward from a position in the second direction (for example, the longitudinal direction) of the medical image 700, and approaches the central position 01 of the medical image 700 in the second direction.

In the present application, adjusting the position that is in the subject 10 to undergo detection and that corresponds to the imaging data obtained by the scanning device 1 not only enables the image 701 of the predetermined site 11 to be more easily observed, but also enables the image 701 of the predetermined site 11 (for example, a blood vessel) after pressing to remain inside the medical image 700 when an operation of pressing the subject to undergo detection is performed, thereby preventing the image 701 of the predetermined site 11 from being outside the medical image 700, so that a detection result can be improved.

In the present application, the medical imaging system 100 shown in FIG. 1 includes suitable hardware, software, or a combination thereof for supporting medical imaging (i.e., enabling the acquisition of data for use in generating and/or rendering images during a medical imaging examination). For example, the medical imaging system 100 may be an ultrasound imaging system or a magnetic resonance system configured to generate and/or render ultrasound images.

As shown in FIG. 1, the medical imaging system 100 may further include a display 114.

The display 114 may be configured to display images (for example, via a screen). In some cases, the display 114 may also be configured to at least partially generate the displayed image. In addition, the display 114 may further support user input/output. For example, in addition to images, the display 114 may further provide (for example, via the screen) user feedback (for example, information related to the system, the functions and settings thereof, etc.). The display 114 may further support user input (for example, via user controls 118) to, for example, allow control of medical imaging. User input can involve controlling the display of images, selecting settings, specifying user preferences, requesting feedback, etc.

In some examples, the medical imaging system 100 may further incorporate additional and dedicated computing resources, such as one or more computing systems 120. In this regard, each computing system 120 may include circuits, interfaces, logic, and/or code suitable for processing, storing, and/or communicating data. The computing system 120 may be a specialized device configured for use specifically in conjunction with medical imaging, or it may be a general-purpose computing system (for example, a personal computer, server, etc.) that is set up and/or configured to perform the operations described below with respect to computing system 120. The computing system 120 may be configured to support the operation of the medical imaging system 100, as described below. In this regard, various functions and/or operations can be offloaded from the imaging system, which may simplify and/or centralize certain aspects of processing to reduce costs (by eliminating the need to add processing resources to the imaging system).

The computing system 120 may be set up and/or arranged for use in different ways. For example, in some specific implementations, a single computing system 120 may be used; and in other specific implementations, multiple computing systems 120 are configured to work together (e.g., on the basis of a distributed processing configuration), or individually. Each of the computing systems 120 is configured to process specific aspects and/or functions, and/or to process data only for a specific medical imaging system 100.

In some examples, the computing system 120 may be local (for example, co-located with one or more medical imaging systems 100, such as within the same facility and/or the same local network); and in other specific embodiments, the computing system 120 may be remote, and thus accessible only by means of a remote connection (for example, by means of the Internet or other available remote access technologies). In particular specific implementations, the computing system 120 may be configured in a cloud-based manner and may be accessed and/or used in a substantially similar manner to accessing and using other cloud-based systems.

Once data is generated and/or configured in the computing system 120, the data can be copied and/or loaded into the medical imaging system 100. This can be done in different ways. For example, the data may be loaded via a directed connection or link between the medical imaging system 100 and the computing system 120. In this regard, communication between the different components of the setup can be performed using available wired and/or wireless connections and/or according to any suitable communication (and/or networking) standards or protocols. Alternatively or additionally, the data may be loaded indirectly into the medical imaging system 100. For example, the data may be stored in a suitable machine-readable medium (for example, a flash memory card) and then loaded into the medical imaging system 100 using the machine-readable medium (on-site, for example, by a user of the system (such as an imaging clinician) or authorized personnel); or the data may be downloaded to a locally communicative electronic device (for example, a laptop) and then said electronic device is used on-site (for example, by a user of the system or authorized personnel) to upload the data to the medical imaging system 100 by means of a direct connection (for example, a USB connector).

In operation, the medical imaging system 100 may be used to generate and present (for example, render or display) images during a medical examination and/or used in conjunction therewith to support user input/output. The images can be 2D, 3D, and/or 4D images. The particular operations or functions performed in the medical imaging system 100 to facilitate the generation and/or presentation of images depend on the type of system (for example, the means used to obtain and/or generate the data corresponding to the images). For example, in ultrasound imaging, the data is based on the emitted ultrasound signal and the echo ultrasound signal 0.

FIG. 8 is a schematic diagram of an ultrasound imaging system according to an example of the present application. As shown in FIG. 8, an ultrasound imaging system 200 may be configured to provide ultrasound imaging, and may therefore include suitable circuitry, interfaces, logic, and/or code for performing and/or supporting ultrasound imaging related functions. The ultrasound imaging system 200 may correspond to the medical imaging system 100 of FIG. 1.

The ultrasound imaging system 200 includes, for example, a transmitter 202, an ultrasound probe 204 (corresponding to the foregoing scanning device 1), a transmitting beamformer 210, a receiver 218, a receiving beamformer 220, an RF processor 224, an RF/IQ buffer 226, a user input module 230, a signal processor 240 (corresponding to the foregoing control apparatus 3), an image buffer 250, a display system 260 (display), a file 270, and an indication apparatus 280 (corresponding to the foregoing indication apparatus 2). The indication apparatus 280 may be disposed on the ultrasound probe 204, and communicates with the signal processor 240. Under the control of the signal processor 240, the indication apparatus 280 can project a light beam onto a predetermined surface, so as to form a pattern by transmitting the light beam through the predetermined surface, thereby performing the solution of any of the examples of the present application.

The transmitter 202 may include suitable circuitry, interfaces, logic, and/or code operable to drive the ultrasound probe 204. The ultrasound probe 204 may include an array of two-dimensional (2D) piezoelectric elements. The ultrasound probe 204 may include a set of transmitting transducer elements 206 and a set of receiving transducer elements 208 that typically form the same element. In some embodiments, the ultrasound probe 204 may be operable to acquire ultrasound image data covering at least a substantial portion of an anatomical structure (such as the heart or any suitable anatomical structure).

The transmitting beamformer 210 may include suitable circuitry, interfaces, logic, and/or code that is operable to control the transmitter 202, and the transmitter 202 drives the set of transmitting transducer elements 206 through a transmitting subaperture beamformer 214 to transmit ultrasound emission signals into a region of interest (for example, a person, animal, subsurface cavity, physical structure, etc.). The emitted ultrasound signal can be backscattered from structures in the subject of interest (for example, blood cells or tissue) to produce echoes. The echo is received by the receiving transducer element 208.

The set of receiving transducer elements 208 in the ultrasound probe 204 may be operated to convert the received echo to an analog signal for subaperture beam formation through a receiving subaperture beamformer 216, which is then transmitted to the receiver 218. The receiver 218 may include suitable circuitry, interfaces, logic, and/or code that is operable to receive signals from the receiving subaperture beamformer 216. The analog signal can be transferred to one or more of a plurality of A/D converters 222.

The plurality of A/D converters 222 may include suitable circuitry, interfaces, logic, and/or code that is operable to convert the analog signal from the receiver 218 to a corresponding digital signal. The plurality of A/D converters 222 are provided between the receiver 218 and the RF processor 224. Nevertheless, the present application is not limited in this regard. Thus, in some embodiments, the plurality of A/D converters 222 may be integrated within the receiver 218.

The RF processor 224 may include suitable circuitry, interfaces, logic, and/or code that is operable to demodulate the digital signals output by the plurality of A/D converters 222. According to one embodiment, the RF processor 224 may include a complex demodulator (not shown) that is operable to demodulate the digital signal to form an I/Q data pair representing the corresponding echo signal. The RF or I/Q signal data can then be transferred to the RF/IQ buffer 226. The RF/IQ buffer 226 may include suitable circuitry, interfaces, logic, and/or code that is operable to provide temporary storage of RF or I/Q signal data generated by the RF processor 224.

The receiving beamformer 220 may include suitable circuitry, interfaces, logic, and/or code that may be operable to perform digital beamforming processing to, for example, sum and output a beam summing signal for the delay-channel signals received from the RF processor 224 via the RF/IQ buffer 226. The resulting processed information may be the beam summing signal output from the receiving beamformer 220 and transmitted to the signal processor 240. According to some embodiments, the receiver 218, the plurality of A/D converters 222, the RF processor 224, and the beamformer 220 may be integrated into a single beamformer which may be digital. In various embodiments, the ultrasound imaging system 200 includes a plurality of receiving beamformers 220.

The user input device 230 can be used to enter patient data, scan parameters, and settings, and select protocols and/or templates to interact with the AI segmentation processor, so as to select tracking targets, etc. In an illustrative embodiment, the user input device 230 is operable to configure, manage, and/or control the operation of one or more components and/or modules in the ultrasound imaging system 200. In this regard, the user input device 230 is operable to configure, manage, and/or control the operation of the transmitter 202, the ultrasound probe 204, the transmitting beamformer 210, the receiver 218, the receiving beamformer 220, the RF processor 224, the RF/IQ buffer 226, the user input device 230, the signal processor 240, the image buffer 250, the display system 260, and/or the file 270.

For example, the user input devices 230 may include buttons, rotary encoders, touch screens, motion tracking, voice recognition, mouse devices, keyboards, trackballs, cameras, and/or any other devices capable of receiving user commands. In some embodiments, for example, one or more of the user input devices 230 may be integrated into other components (such as the display system 260 or the ultrasound probe 204). As an example, the user input device 230 may include a touch screen display. As another example, the user input device 230 may include an accelerometer, gyroscope, and/or magnetometer attached to and/or integrated with the probe 204 to provide pose and motion recognition of the probe 204, such as identifying one or more probe compressions against the patient's body, predefined probe movements, or tilt operations, etc. Additionally and/or alternatively, the user input device 230 may include image analysis processing to identify the probe pose by analyzing the captured image data.

The signal processor 240 may include suitable circuitry, interfaces, logic, and/or code that is operable to process the ultrasound scan data (for example, the summed IQ signal) to generate an ultrasound image for presentation on the display system 260. The signal processor 240 is operable to perform one or more processing operations based on a plurality of selectable ultrasound modalities on the acquired ultrasound scan data. In an illustrative embodiment, the signal processor 240 is operable to perform display processing and/or control processing, etc. As the echo signal is received, the acquired ultrasound scan data can be processed in real-time during the scan session. Additionally or alternatively, the ultrasound scan data may be temporarily stored in the RF/IQ buffer 226 during the scan session and processed in a less real-time manner during online or offline operation. In various embodiments, the processed image data may be presented at the display system 260 and/or may be stored in the file 270. The file 270 can be a local file, a picture archiving and communication system (PACS), or any suitable device for storing images and related information.

The signal processor 240 may be one or more central processing units, microprocessors, microcontrollers, etc. For example, the signal processor 240 may be an integrated component, or may be distributed in various locations. The signal processor 240 may be configured to receive input information from the user input device 230 and/or file 270, generate outputs that may be shown by the display system 260, and manipulate the outputs, etc., in response to the input information from the user input device 230. The signal processor 240 may be capable of executing, for example, any of one or more of the methods and/or one or more sets of instructions discussed herein according to various embodiments.

The ultrasound imaging system 200 may be operated to continuously acquire ultrasound scan data at a frame rate suitable for the imaging situation under consideration. Typical frame rates are in the range of 20 to 220, but can be lower or higher. The acquired ultrasound scan data can be shown on the display system 260 in real-time at a display rate that is the same as the frame rate, or slower, or faster than the frame rate. The image buffer 250 is included to store frames for processing of the acquired ultrasound scan data that are not scheduled for immediate display. Preferably, the image buffer 250 has sufficient capacity to store frames of ultrasound scan data for at least a few minutes. Frames of ultrasound scan data are stored in such a way that they can be easily retrieved therefrom according to their acquisition sequence or time. The image buffer 250 may be embodied in any known data storage medium.

In some specific embodiments, the signal processor 240 may be configured to perform or otherwise control at least some of the functions performed thereby based on user instructions via the user input device 230. As an example, the user may provide voice commands, probe poses, button presses, etc. to issue specific commands such as controlling aspects of automatic strain measurement and strain ratio calculations, and/or provide or otherwise specify various parameters or settings associated therewith, as described in more detail below.

In operation, the ultrasound imaging system 200 may be used to generate ultrasound images, including two-dimensional (2D), three-dimensional (3D), and/or four-dimensional (4D) images. In this regard, the ultrasound system 200 may be operated to continuously acquire ultrasound scan data at a specific frame rate, which may be applicable to the imaging situation discussed. For example, the frame rate can be in the range of 20-70, or can be lower or higher. The acquired ultrasound scan data can be shown on the display system 260 at the same display rate as the frame rate, or slower, or faster than the frame rate. The image buffer 250 is included to store frames for processing of the acquired ultrasound scan data that are not scheduled for immediate display. Preferably, the image buffer 250 has sufficient capacity to store at least a few seconds of frames of ultrasound scan data. Frames of ultrasound scan data are stored in such a way that they can be easily retrieved therefrom according to their acquisition sequence or time. The image buffer 250 may be embodied in any known data storage medium.

In some cases, the ultrasound imaging system 200 may be configured to support grayscale and color-based operations. For example, the signal processor 240 may operate to perform grayscale B-model processing and/or color processing. Grayscale B-model processing may include processing B-model RF signal data or IQ data pairs. For example, the grayscale B-model processing can enable the formation of an envelope of the received beam summing signal by computing the amount (I²+Q²)^{1/2}. The envelope can be subjected to additional B-model processing, such as logarithmic compression to form the display data. The display data can be converted to X-Y format for video display. Scan-converted frames can be mapped to grayscale for display. The B model frame is provided to the image buffer 250 and/or the display system 260. Color processing may include processing color-based RF signal data or IQ data pairs to form frames to cover the B-model frames being provided to image buffer 250 and/or display system 260. Grayscale and/or color processing may be self-adaptively adjusted based on user input (for example, selections from user input device 230), such as for enhancing the grayscale and/or color of a particular region.

Examples of the present application further provide a control method of a medical imaging system, to control a medical imaging system 100.

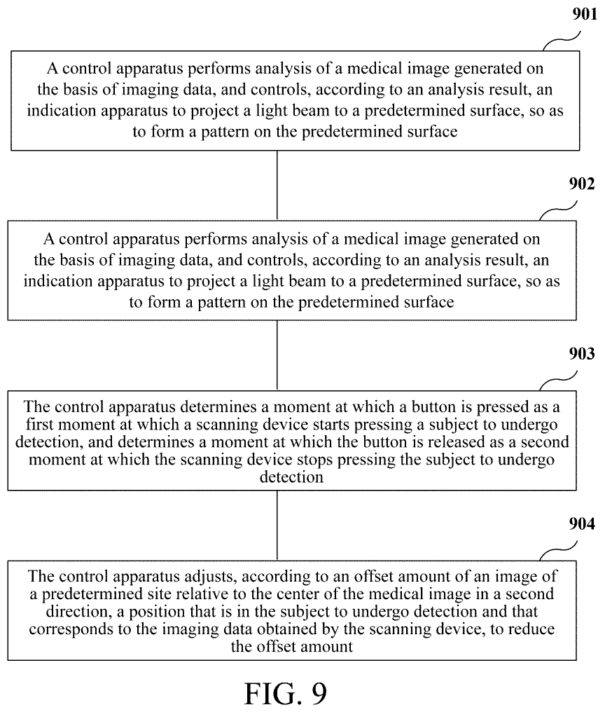

FIG. 9 is a schematic diagram of a control method of a medical imaging system. As shown in FIG. 9, the control method includes:
901. A control apparatus performs analysis of a medical image generated on the basis of imaging data, and controls, according to an analysis result, an indication apparatus to project a light beam to a predetermined surface, so as to form a pattern on the predetermined surface.

In some examples, the analysis includes analysis of a difference between an image of a predetermined site in the medical image and a target, wherein the pattern indicates target movement information of a scanning device to reduce the difference.

In some examples, the difference includes an offset of the image of the predetermined site relative to a central position of the medical image, and the target movement information includes first target translation information for reducing the offset.

In some examples, the difference includes a difference between a size difference, between a first size in a first direction and a second size in a second direction of the image of the predetermined site, and a second threshold, and the target movement information includes second target rotation information for reducing the difference.

In some examples, the control apparatus further performs real-time analysis while the scanning device is being moved, and controls, according to a real-time analysis result, the indication apparatus to form a real-time pattern.

In some examples, the analysis result includes a detection result of detection performed on the medical image, when the detection result is a first result, the pattern is a first pattern, when the detection result is a second result, the pattern is a second pattern, and the first pattern and the second pattern are different in at least one of shape, color, and position.

In some examples, as shown in FIG. 9, the control method further includes:
902. After the detection performed on the basis of the medical image is completed, the control apparatus further controls the indication apparatus to project a light beam onto the predetermined surface to form a movement indication pattern on the predetermined surface, wherein the movement indication pattern indicates a second target translation direction of the scanning device.

In some examples, an end point of the movement indication pattern in the second target translation direction is used to indicate a target position for the scanning device moving in the second target translation direction.

In some examples, the predetermined site is a blood vessel, the analysis includes analysis of a health condition of the blood vessel, and the pattern indicates the health condition. In some examples, as shown in FIG. 9, the control method further includes:
903. The control apparatus determines a moment at which a button is pressed as a first moment at which the scanning device starts pressing a subject to undergo detection, and determines a moment at which the button is released as a second moment at which the scanning device stops pressing the subject to undergo detection.

In this way, the control apparatus performs detection according to the medical image at the first moment and the medical image at the second moment.

In some examples, as shown in FIG. 9, the control method further includes:
904. The control apparatus adjusts, according to an offset amount of the image of the predetermined site relative to the center of the medical image in the second direction, a position that is in the subject to undergo detection and that corresponds to the imaging data obtained by the scanning device, to reduce the offset amount.

The examples of the present application further provide a computer-readable program, wherein the program, when executed, causes a computer to perform, in a medical imaging system, the control method described in the foregoing examples.

The examples of the present application further provide a storage medium storing a computer-readable program, wherein the computer-readable program causes a computer to perform, in a medical imaging system, the control method described in the foregoing examples.

The above examples merely provide illustrative descriptions of the examples of the present application. However, the present application is not limited thereto, and appropriate variations may be made on the basis of the above examples. For example, each of the above examples may be used independently, or one or more among the above examples may be combined.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

1. An ultrasonic imaging system, comprising:
a probe configured to scan a predetermined site of a subject to undergo detection, to obtain imaging data containing information about the predetermined site, the probe comprising:
a transducer configured to transmit and receive an ultrasound signal;
a matching layer configured to have an acoustic impedance between a tissue to be imaged and a material of the transducer;
a damping block configured to absorb ultrasound energy; and
an indicator configured to project a light beam; and
a controller configured to analyze a medical image generated based on the imaging data, and control, according to an analysis result, the indicator to project a light beam to a predetermined surface to form a pattern on the predetermined surface.

2. The medical imaging system according to claim 1, wherein
the analysis comprises analysis of a difference between an image of the predetermined site in the medical image and a target, and
the pattern indicates target movement information for the probe to reduce the difference.

3. The medical imaging system according to claim 2, wherein
the difference comprises an offset of the image of the predetermined site relative to a central position of the medical image, and
the target movement information comprises first target translation information for reducing the offset.

4. The medical imaging system according to claim 2, wherein
the difference comprises a difference between a size difference, between a first size in a first direction and a second size in a second direction of the image of the predetermined site, and a second threshold, and
the target movement information comprises second target rotation information for reducing the difference.

5. The medical imaging system according to claim 2, wherein
the controller is further configured to provide real-time analysis while the probe is being moved, and controls, according to a real-time analysis result, the indicator to form a real-time pattern.

6. The medical imaging system according to claim 1, wherein
the analysis result comprises a detection result obtained by performing detection on the medical image,
based on the detection result being a first result, the pattern is a first pattern,
based on the detection result being a second result, the pattern is a second pattern, and
the first pattern and the second pattern are different in at least one of shape, color, and position.

7. The medical imaging system according to claim 1, wherein
after detection performed based on the medical image is completed,
the controller further is further configured to control the indicator to project a light beam to the predetermined surface, to form a movement indication pattern on the predetermined surface, and
the movement indication pattern indicates a second target translation direction of the probe.

8. The medical imaging system according to claim 7, wherein
an end point of the movement indication pattern in the second target translation direction is used to indicate a target position for the probe moving in the second target translation direction.

9. The medical imaging system according to claim 1, wherein
the predetermined site is a blood vessel, the analysis comprises analysis of a health condition of the blood vessel, and the pattern indicates the health condition.

10. The medical imaging system according to claim 1, wherein
the probe has a button, and
the controller is configured to:
determine a moment at which the button is pressed as a first moment at which the probe starts pressing the subject to undergo detection, and
determine a moment at which the button is released as a second moment at which the probe stops pressing the subject to undergo detection,
wherein the controller performs detection according to the medical image at the first moment and the medical image at the second moment.

11. The medical imaging system according to claim 1, wherein
the controller is configured to adjust, according to an offset amount of the image of the predetermined site relative to the center of the medical image in a second direction, a position that is in the subject to undergo detection and that corresponds to the imaging data obtained by the probe, to reduce the offset amount.

12. A method of controlling a medical imaging system, the medical imaging system comprising: a scanning device configured to scan a predetermined site of a subject to undergo detection, to obtain imaging data containing information about the predetermined site; an indicator configured to emit light; and a controller;
the method comprising performing analysis of a medical image generated based on the imaging data, and controlling, according to an analysis result, the indicator to project a light beam to a predetermined surface, to form a pattern on the predetermined surface.

13. The control method of a medical imaging system according to claim 12, wherein
the analysis comprises analysis of a difference between an image of the predetermined site in the medical image and a target, and
the pattern indicates target movement information for the scanning device to reduce the difference.

14. The control method of a medical imaging system according to claim 13, wherein
the difference comprises an offset of the image of the predetermined site relative to a central position of the medical image, and
the target movement information comprises first target translation information for reducing the offset.

15. The control method of a medical imaging system according to claim 13, wherein
the difference comprises a difference between a size difference, between a first size in a first direction and a second size in a second direction of the image of the predetermined site, and a second threshold, and
the target movement information comprises second target rotation information for reducing the difference.

16. The control method of a medical imaging system according to claim 13, wherein
the controller further performs real-time analysis while the scanning device is being moved, and controls, according to a real-time analysis result, the indicator to form a real-time pattern.

17. The control method of a medical imaging system according to claim 12, wherein
the analysis result comprises a detection result obtained by performing detection on the medical image,
when the detection result is a first result, the pattern is a first pattern,
when the detection result is a second result, the pattern is a second pattern, and
the first pattern and the second pattern are different in at least one of shape, color, and position.

18. The control method of a medical imaging system according to claim 12, wherein
the control method further comprises:
after detection performed based on the medical image is completed,
the controller further controls the indicator to project a light beam to the predetermined surface, to form a movement indication pattern on the predetermined surface, and
the movement indication pattern indicates a second target translation direction of the scanning device.

19. The control method of a medical imaging system according to claim 18, wherein
an end point of the movement indication pattern in the second target translation direction is used to indicate a target position for the scanning device moving in the second target translation direction.

20. An imaging system, comprising:
a scanner configured to scan a predetermined site of a subject to undergo detection, to obtain imaging data containing information about the predetermined site, the scanner comprising an indicator configured to project a light beam; and
a controller configured to analyze a medical image generated based on the imaging data, and control, according to an analysis result, the indicator to project a light beam to a predetermined surface to form a pattern on the predetermined surface.
